(19) United States
(12) Patent Application Publication
(10) Pub. No.: US 2026/0096739 A1
(43) Pub. Date: Apr. 9, 2026

(54) Handheld Medical Eddy Current Induction Damping Sensor

(71) Applicant: StrokeDx, Inc., Culver City, CA (US)

(72) Inventors: Shane S. Shahrestani, Yorba Linda, CA (US); Alexander M. Ballatori, Rushville, NY (US); Brian L. Nguyen, Pasadena, CA (US)

(73) Assignee: StrokeDx, Inc., Culver City, CA (US)

(21) Appl. No.: 19/340,631

(22) Filed: Sep. 25, 2025

Related U.S. Application Data
(60) Provisional application No. 63/703,513, filed on Oct. 4, 2024.

(51) Int. Cl. A61B 5/0536; A61B 5/00; A61B 5/06; A61B 34/20

(52) U.S. Cl. CPC A61B 5/0536; A61B 5/067; A61B 5/4887; A61B 5/742; A61B 34/20; A61B 2560/0223

(57) ABSTRACT

PROBLEM TO BE SOLVED: To easily perform the regulating work of resonance frequency at a low cost by mounting a socket or connector on a coil conductor, and attachably and detachably mounting a capacitor board having a capacitor thereon on the socket or connector.

SOLUTION: An RF coil for MRI 100 comprises to coil conductor 1 having the fixed capacitor 2, and the socket 3 provided on a part of the coil conductor 1. The capacitor board 10 having a fixed capacitor 11 thereon is mounted on the coil conductor 1 attachable to and detachable from the socket 3. The used fixed capacitor 2 shares the fixed portion of the resonance frequency, and the fixed capacitor 11 is provided in order to share the variable portion of the resonance frequency, The socket 3 has a number of pins raised thereon, and the pins are inserted to a number of holes of the capacitor board 10. whereby the coil conductor 1 is electrically connected to the capacitor 11.

### CROSS-REFERENCES TO RELATED APPLICATIONS

This application claims the benefit of U.S. Provisional Application No. 63/703,513, filed 4 Oct. 2024, the contents of which are hereby incorporated by reference in its entirety for all purposes.

### STATEMENT AS TO RIGHTS TO INVENTIONS MADE UNDER FEDERALLY SPONSORED RESEARCH AND DEVELOPMENT

Not applicable.

### BACKGROUND

### 1. Field of the Invention

The present application generally relates to measuring, for surgical diagnostic purposes, in vivo body composition using a probe whose position is determined with accelerometers or gyroscopes. Specifically, the application is related to a handheld device with a magnetic coil electronically coupled with a resonant frequency circuit that can be scanned around a body to locate a hemorrhagic or ischemic anomaly and assist in treating it.

### 2. Description of the Related Art

Diagnosing a possible stroke in a patient is frequently difficult because the stroke victim is often unconscious, and there are few, if any, visible indications of what is going on inside the patient's cranium. Further, a treating physician is under time pressure to proceed with a course of treatment. Each minute that passes untreated can result in greater and greater brain damage, often permanent. A wrong treatment can be worse.

Ideally, treatment for a stroke should occur as early as possible. Staff at a receiving emergency room should begin preparing even before a patient arrives by ambulance. There are different types of treatments for different types of strokes, and staff should prepare for the most probable of contingencies. Staging equipment, supplies, and drugs for different contingencies takes time and can waste sterilization and consumables.

The wrong treatment may be fatal. Treatment for an ischemic stroke involves administering blood thinners. However, if the patient is actually experiencing a hemorrhagic stroke, then the blood thinners may worsen the problem by letting internal bleeding within the brain continue unabated by the blood's natural coagulation properties.

In certain emergency circumstances, an external ventricular drain (EVD) is required to remove excess cerebrospinal fluid (CSF) or blood that is pooling in a patient's brain. It includes a catheter through which the fluid flows to outside the cranium, thereby relieving internal pressure. A treating physician typically inserts an EVD by measuring across the patient's head-with a small ruler-to where an incision should be made. He or she then bores a hole in the cranium and then inserts an EVD deep into the brain, typically into a ventricle.

After initial treatment, it is important to monitor a stroke patient to determine if things are getting better, worse, or staying the same. While a patient could be admitted and treated for one type of stroke, drugs and other intervention could cause another.

Currently, computed tomography (CT) and magnetic resonance imaging (MRI) are the two gold standards for measuring, diagnosing, and monitoring brain health for stroke patients. When available, they help immensely by providing a treating physician with a view inside the patient's brain. And they are employed repeatedly on the patient so as to track progress, or lack thereof.

However, these devices are large, expensive, often require specialized staff, and are mostly restricted to larger hospital systems. CT scans give a non-negligible radiation dose, which adds to the patient's cancer risk. Neither can be used continuously, or even frequently, let alone on demand at a patient bedside.

There is a need in the art for portable and less expensive systems that can detect a fluidic anomaly within a patient's head or other body parts and monitor it over time, among other things.

### BRIEF SUMMARY

A handheld medical sensor for scanning over a patient's body, primarily the patient's head, employs a coil and a resonant resistive, inductive, and capacitive (RLC) circuit to detect conductive fluid, or the lack thereof, within the body using eddy currents. A user holds the device against the body and sweeps over it. The device calculates its 3-dimensional (3D) location and orientation in space from an inertial measurement unit (IMU) along with resonance and damping measurements from the coil. By sensing physical contact with the patient, the device can map the body surface and show, on a display, anomalies with respect to a digital outline of the patient's body. The location and orientation of the sensor itself can also be displayed along with a depiction of the body part and/or overlayed computed tomography (CT) or magnetic resonance imaging (MRI) image.

The sensor senses anomalies by using a frequency counter and power meter that are electrically connected with the RLC circuit. The frequency counter and power meter generate outputs that are converted by computer to measured values. The measured values can be parallel resistances (R_{P}) with respect to an equivalent circuit, natural frequencies, or other measurements. The outputs reflect changes in the resonant frequency of the RLC-coil circuit when the coil magnetically induces eddy currents in nearby tissue. There can be multiple coils, RLC circuits, and frequency counters, with multiple coils being coaxial with one another.

When an anomaly is determined, the sensor can be affixed directly over it on the body or at a location where there is access to the anomaly. The sensor's handle may be removed from its base. If a physician decides that the anomaly needs drainage, then an incision is made in the scalp at the location of the sensor base, and an external ventricular drain (EVD) introduced through a hole in the sensor base. The EVD catheter itself or its stylet can have metal rings or other conductive features. As the EVD catheter progresses through the sensor, its coil can detect the number of metal rings that pass through it and determine how far the EVD catheter sticks through. The estimated location of the end of the EVD catheter can then be shown on the display.

Some embodiments of the invention are related to a handheld inductive sensor apparatus for body diagnostics, including an electrical coil, a resistive, inductive, and capacitive (RLC) circuit electrically connected with the coil, a frequency counter electrically connected with the RLC circuit, an inertial measurement unit (IMU) rigidly connected with the coil, and a computer processor operatively connected with a machine-readable tangible non-transitory medium embodying information indicative of instructions for causing the computer processor to perform operations including generating a measured value based on an output from the frequency counter, calculating a position and an orientation of the coil with respect to a body part of a subject based on output from the IMU, and associating the position and the orientation with the measured value.

The operations can further include the processor accessing, from a database, file, or otherwise, a 3-dimensional (3D) model representing the body part, and rendering, using the model, an image of the body part along with a graphic representing the position and the orientation of the coil with respect to the body part. The model can be derived from a computerized tomography (CT) scan or a magnetic resonance imaging (MRI) scan of the body part of the subject.

The operations can further include depicting, in the image, an internal feature of the body part based on the MRI scan or CT scan. The model can be a representatively standard body part of the subject's species or from measurements of the particular subject.

The operations can include determining an anomaly in the body part based on the measured value, and placing, in the image of the body part, an indicator of the anomaly. They can include receiving a calibration command from a user while the coil is placed against the body part at a predetermined location and at a predetermined orientation.

The apparatus can include a housing for the coil, the housing including an aperture extending through the coil. The operations can further include receiving a command from the user or software to switch modes, determining an extent that a surgical tool projects through the aperture based on the measured value, and depicting, on the image, a depth of the surgical tool within the body part.

A surgical kit can include the apparatus as well as the surgical tool, the surgical tool having conductive rings spaced along a length of a catheter. The operations can further include counting a number of rings that have passed through the coil, and calculating the extent based on the counting. Instead of or in addition to the conductive rings, the surgical tool can have different metals spaced along a length of a catheter, and the operations can further include determining at least one of the metals that has passed through the coil based on a conductivity of the at least one of the metals, and calculating the extent based on the determining. Instead of or in addition to the conductive rings and different metals, the surgical tool can have an increasing amount of conductive material spaced along a length of a catheter, and the operations can further include determining an amount of conductive material that has passed through the coil, and calculating the extent based on the determining.

The apparatus can further include a proximity sensor connected with the processor, and the operations can further include determining whether the coil abuts the body part based on an output from the proximity sensor, and locating the body part in 3-dimensional (3D) space based on the determination. The operations can further include interpolating a surface in 3D space of the body surface based on the locating of the body part.

The apparatus can include a removable hand grip connected with the coil. The operations can further include indicating, based on the measured value, that the coil has moved away from the subject.

The body part can be a head of the subject. The apparatus can further include a housing for the coil, the housing including a groove, notch, or other recess configured to mate with a nasal bridge of the subject during calibration.

Some embodiments are related to a method of manufacturing a handheld inductive sensor for body diagnostics, the method including providing an electrical coil, connecting a resistive, inductive, and capacitive (RLC) circuit electrically with the coil, electrically connecting a frequency counter with the RLC circuit, rigidly connecting an inertial measurement unit (IMU) with the coil, and operatively connecting a computer processor with a machine-readable tangible non-transitory medium embodying information indicative of instructions for causing the computer processor to perform operations including generating a measured value based on an output from the frequency counter, calculating a position and an orientation of the coil with respect to a body part of a subject based on output from the IMU, and associating the position and the orientation with the measured value.

Some embodiments are related to a method of using a handheld inductive sensor for body diagnostics, the method including providing a sensor having an electrical coil, a resistive, inductive, and capacitive (RLC) circuit electrically connected with the coil, a frequency counter electrically connected with the RLC circuit, an inertial measurement unit (IMU) rigidly connected with the coil, and a computer processor, generating a measured value based on an output from the frequency counter, calculating, using the computer processor, a position and an orientation of the coil with respect to a body part of a subject based on output from the IMU, and associating, using the computer processor, the position and orientation with the measured value.

### BRIEF DESCRIPTION OF THE DRAWINGS

FIG. 1 illustrates a perspective side view of a handheld sensor and computer in accordance with an embodiment.

FIG. 2 is a circuit diagram of an eddy current coil sensor in accordance with an embodiment.

FIG. 3A illustrates a handheld sensor scanning a head at a top position in accordance with an embodiment.

FIG. 3B illustrates the handheld sensor of FIG. 3A scanning a head at a front position in accordance with an embodiment.

FIG. 3C illustrates the handheld sensor of FIG. 3A scanning a head at a left position in accordance with an embodiment.

FIG. 3D illustrates the handheld sensor of FIG. 3A scanning a head at a right position in accordance with an embodiment.

FIG. 4 illustrates a monitor displaying an image of a head along with a graphic representing the position and the orientation of the coil with respect to the head overlayed with a magnetic resonance imaging (MRI) image in accordance with an embodiment.

FIG. 5 illustrates a handheld sensor scanning a head at a diagonal angle in accordance with an embodiment.

FIG. 6 depicts MRI images overlayed with a contemporaneous position of the sensor of FIG. 5 in accordance with an embodiment.

FIG. 7 illustrates a top side perspective view of a handheld sensor with an aperture for a surgical tool in accordance with an embodiment.

FIG. 8 illustrates a handheld sensor with a surgical tool passing therethrough in accordance with an embodiment.

FIG. 9 depicts MRI images overlayed with a contemporaneous position of the surgical tool of FIG. 8 in accordance with an embodiment.

FIG. 10 illustrates a side view of an external ventricular drain (EVD) catheter with metal rings in accordance with an embodiment.

FIG. 11 illustrates a side view of an EVD catheter with rings of different metals in accordance with an embodiment.

FIG. 12 illustrates a side view of an EVD catheter with increasing metal thickness along its length, thinner distally, thicker proximally, in accordance with an embodiment.

FIG. 13 shows a block diagram of a computer system in accordance with an embodiment.

FIG. 14 is a flowchart illustrating a process in accordance with an embodiment.

FIG. 15 is a flowchart illustrating a process in accordance with an embodiment.

### DETAILED DESCRIPTION

A handheld medical diagnostic device for scanning a patient for internal abnormal conductive fluid concentrations is described in which a coil is connected through a resistive, inductive, and capacitive (RLC) resonant circuit to a frequency counter and/or a power meter, and the coil's position and orientation on a body is tracked in real-time by an inertial measurement unit (IMU). The coil magnetically induces eddy currents in nearby tissue, the eddy currents changing the resonant frequency and damping of the circuit. Readings from a frequency counter that detects resonant frequencies, and/or a power meter to determine damping, are recorded and interpreted to identify anomalies.

To scan the brain for a stroke, the device can be moved from location to location on a patient's head. Measurements from the left and right hemispheres can be compared with each other to determine anomalous fluid concentrations. Measurements may also be taken over time to track a patient's recovery or downward trend, or they may be compared with averaged readings from other individuals. A microcontroller within or external to the device may be used to stream real-time data to a computer and display. The display can render graphics depicting the sensor's position with respect to the body, the body part of interest itself, and/or any anomalies found within the body.

The sensor device can be parked at a particular location on a subject's body where an anomaly is found. The device's handle can be removed for better access to the location on the body and expose an aperture through the sensor coil. The aperture, preferably centered within the coil, can be used for marking the skin or to aid a physician's use of other diagnostics or medical tools.

In the case of a subject's head, the sensor can be placed over an area where hydrocephalus, a condition where excess cerebrospinal fluid is pooled in the brain, is detected. An external ventricular drain (EVD) may be inserted through the aperture into the brain. The sensor's coil counts metal rings or determines differences in metals or the amount of metal longitudinally along the EVD catheter in order to determine how far the EVD catheter extends through the coil and into the brain. A graphical representation of the surgical tool's location and extent of its distal tip with respect to the brain can be rendered on a display. It can be overlayed upon an MRI or CT scan image.

These devices, their manufacture, and procedures for use will be explained and detailed before

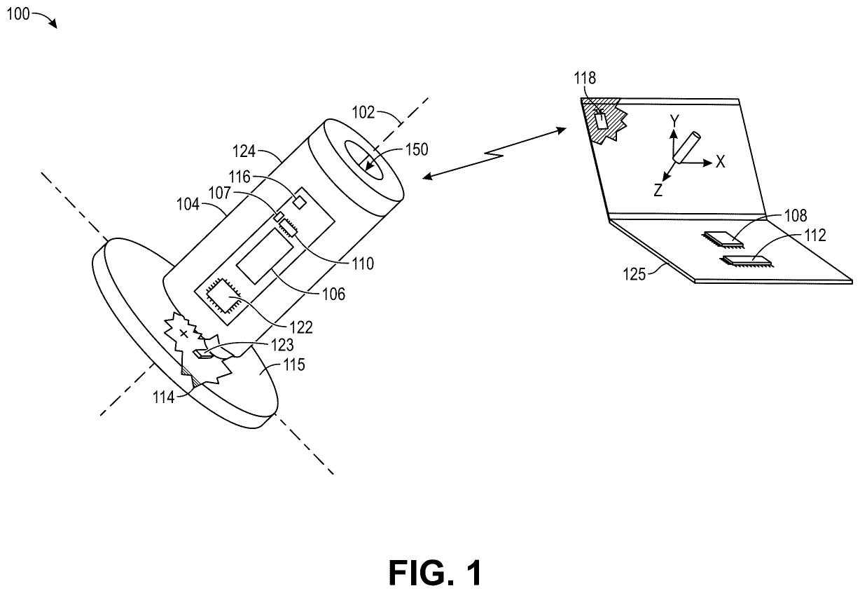

FIG. 1 illustrates a perspective top side view of a handheld sensor and a wirelessly connected computer in system 100.

Handheld sensor 104 comes in two parts: coil housing 115 and handle 124. Coil housing 115 minimally includes electrical coil 114. In the exemplary embodiment, other coils, larger and smaller, are coaxial with the coil. Center axis 102 is normal to the coils and passes through its geometric center or electrical center equivalent.

Coil housing 115 is shown connected with handle/housing 124, which acts as a hand grip for positioning the coil around a subject. Handle 124 can be removed from coil housing 115. Both coil housing 115 and handle 124 have aperture 150 passing through them, forming a lumen around axis 102.

Some embodiments do not include an aperture through the handle or coil, and some include an aperture passing through the coil but not the coil. Some embodiments include just one coil, some two, and some more that two that are coaxial with each other.

Handle 124 houses inertial measurement unit (IMU) 122, which, when the handle is connected with coil housing 115, is rigidly connected with and precisely positioned with respect to the center of sensor coil 114. While not in the exemplary embodiment, the IMU can be centered along axis in some embodiments. It can be aligned with a plane of the coil.

Also housed within handle 124 is resistive, inductive, and capacitive (RLC) circuit 106, which is connected with electrical coil 114. Further frequency counter 110, which is connected with RLC circuit 106, shares the housing. Power meter 107 is also connected to RLC circuit 106 and within the housing. The frequency counter and power meter generate outputs that can be used to determine whether there is conductive material near the coil. That is, coil 114, RLC circuit 106, and frequency counter 110 and/or power meter 107 operate as an eddy current detector that sets up a magnetic field and detects countering magnetic fields that occur in nearby conductive materials.

The outputs of frequency counter 110 and power meter 107 pass to radio frequency (RF) transmitter 116, which communicates with RF receiver 118 in a nearby computer. The outputs, which are digital, are timestamped together with or otherwise associated with outputs from IMU 122, which indicate position and orientation. The outputs from the IMU are also communicated through RF transmitter and RF receiver 118 to the computer.

Computer 125 hosts computer processor 108 that executes instructions from memory 112. The instructions can generate measured values based on the outputs from frequency counter 110, power meter 107, and IMU 122. From a calibrated or base position of a body part, the computer can calculate a position and orientation of coil 114 and sensor 104 with respect to the body part. For example, the IMU can output a longitude and latitude on the head.

Proximity sensor 123, located at the distal end of sensor 104 near coil 114, can detect when the coil touches or is close to a subject. In the exemplary embodiment, the proximity sensor is a mechanical switch that switches when touched. Output from the proximity sensor is funneled through transmitter 116 and can be used for calibration, to affix a position on exactly where the body is in 3-dimensional (3D) space, and/or to flag when the sensor is not against a body part and so data should be ignored.

In some embodiments, a proximity sensor may use non-touch technologies, such as acoustic waves, electrical capacitance, or optics. An optical sensor can determine if, and how fast, the device is swiped over the surface of the subject, much like an optical mouse can determine its speed and direction over a surface.

In some embodiments, the coil itself can be used as a proximity sensor, as the impedance of the circuit changes drastically when lifted away from a body. For example, if the frequency or impedance increases past a predetermined threshold, then it can be presumed that the coil has been lifted from the body.

The computational components can be remote from the RLC circuit, as shown in the exemplary embodiment, or collocated within the same housing. Outputs from the frequency counter and/or power meter can be associated with the position and orientation of the coil onboard the sensor or offboard in a separate computing device. Calibration of the IMU with respect to a subject's head, limb, torso, or other body part of interest can be handled onboard or offboard

The inertial measurement unit (IMU) generates quaternion data representative of the sensor device's orientation in three-dimensional space. This quaternion data is processed by a computational algorithm to derive Euler angles, specifically roll, pitch, and yaw, for orienting a virtual representation.

“Neuronavigation” is determining and communicating position and orientation around the dome of a subject's head for purposes of assessing a brain, or as otherwise known in the art. Neuronavigation can include rendering a representation of the sensor with respect to the subject's head.

For system calibration, the handheld device can be positioned with a designated fiducial marker aligned with a patient's midline and facial plane. This alignment establishes a known, initial spatial relationship between the device and the patient's anatomy, which corresponds to a specific orientation of the internal IMU components.

The handheld can be placed on top of the patient's head, assuming the patient sits upright, and the yaw value is recorded from the IMU. This recorded yaw measurement serves as a reference for the virtual environment. Subsequently, this reference yaw is applied to rotate a 3D head model within a visualization display, thereby aligning the virtual representation with the physical orientation of the patient's head.

Multiple points on a head can be used for calibration, such as the bridge of a nose, top of a head, and left ear. An ordered sweep pattern can be used for calibration. A sterile cap, with fiducial markings, can be used for calibration.

From this calibrated state, all subsequent latitudes, longitudes, and other rotations are visualized relative to the established reference point. The system's visualization protocol maintains the assumption that a projected cylinder, representing a tool or instrument, continuously intersects with the surface of the 3D head model during operation, unless otherwise flagged. For example, if readings indicate that no conductive material at all is nearby, and that the device is in free space, then the visualization protocol may signify that the tool is not active.

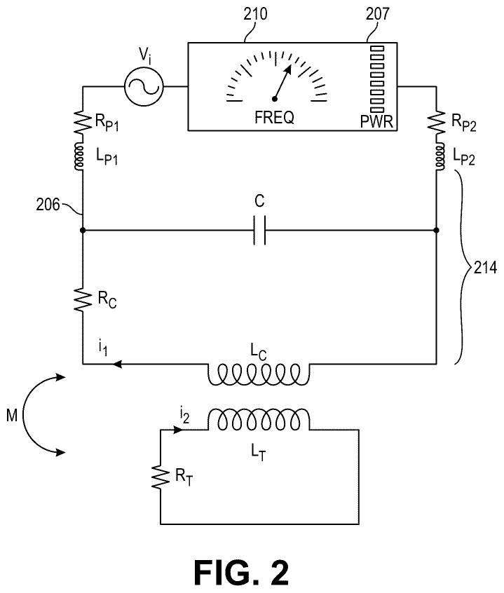

FIG. 2 is a circuit diagram of an eddy current coil sensor. The coil in the sensor is modeled as having current i₁ and producing a magnetic field. This creates magnetic coupling M between it and a target.

The target is a human head, here modeled as a flat, two-layer structure (at figure bottom) with its own current i₂ and induced magnetic field. Single current i₂ models the sum of myriad tiny eddy currents swirling in conductive tissue. Note that currents i₁ and i₂ are in opposite directions, just as eddy currents would establish an opposite direction in order to counter the magnetic field. The head model includes an idealized resistor R_{T} (target) in series with an idealized inductor L_{T}.

RLC circuit 206 includes model of coil 214 with current i₁ running through inductor Lc with resistance Rc and capacitance C. The coil is connected to a voltage source providing Vi alternating current.

Frequency counter 210 and power meter 207 are connected with coil 214. Parasitics of connections are modeled as resistances Rₚ₁ and Rₚ₂ and inductances Lₚ₁ and Lₚ₂. The frequency counter detects the resonant frequency of the coil and RLC circuit. The power meter detects power, or power loss. Absolute values, or changes in them, are output from the frequency counter and power meter and converted in an analog-to-digital (A/D) converter. The result is sent to a computer processor.

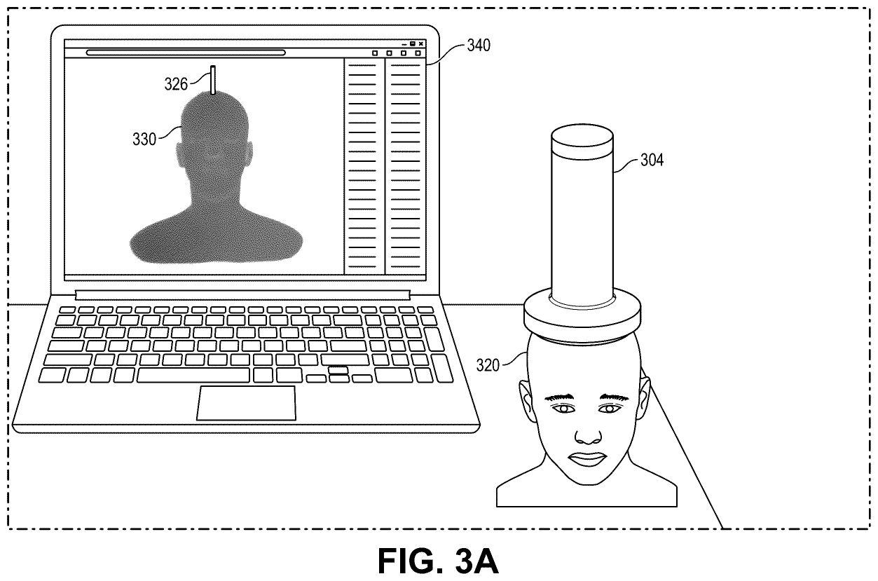

FIGS. 3A-3D illustrate handheld sensor device 304 scanning head 320. The sensor device is held at a known position at the top of a head in order to calibrate it. Other positions can also be used. With the device held at the predetermined position, a user indicates to the system that it is at the calibration point. Accordingly, the IMU may be reset, or an origin point is set in software, or the point is noted as a known position in 3D space.

Computer monitor screen 340 displays an image of head 330 along with a graphic 326 representing the position and the orientation of the coil with respect to the body part. In this case, a cylinder that is normal to the coil is depicted on the screen at its appropriate position on the head in order to represent the device and coil.

The image of head 330 is produced by accessing a 3-dimensional (3D) model representing the head from a file or database and then rendering it on display screen 340. The model is a standard head of a homo sapiens (human). Male, female, or child models can be selected. A 3D model of the actual subject's head may also be used. The model may be derived from an MRI or CT scan, for example, or it may be taken from a scan using the sensor device and proximity sensor.

Proceeding from FIG. 3A to FIG. 3B, sensor 304 is started from the top of the head then swiped down over the forehead down to the nose. This is an alternate or additional location for calibration. A groove recess in the coil housing that is configured to mate with a nasal bridge of a subject may be placed over the bridge of the nose of the subject. A user then may indicate that the sensor device is at the nose bridge calibration site by pressing a button on the computer or sensor. A calibration signal is then sent through the system to reset coordinates or otherwise note the location of the head and sensor in 3D space.

A technical advantage in calibrating the coil location while resting a groove of the housing on a bridge of a nose is that a relatively precise location and orientation of the coil with respect to the head can be obtained. It can work on different head shapes, and it can be applied while a patient is lying down, at least lying supine or on his or her side.

On screen 340, graphic 326 is depicted on the front of the face of head image 330. For calibration, the graphic may be presented in a different shape, color, or size, or another graphic may be positioned as a target at the bridge of the nose on image 330.

Proceeding from FIG. 3B to FIG. 3C, sensor device 304 is slide around the forehead to the subject's left temple. Accordingly, monitor screen 340 displays cylinder graphic 326 on the left temple of head image 330.

From FIG. 3C to FIG. 3D, sensor device 304 is slewed around the rear over the occipital lobe, and to the right side of head 320. Display monitor 340 is updated appropriately. If a patient is lying down instead of upright as shown, then the sensor may be swiped over the front again to get to the other side. The sensor can be swiped in any geometry, even lifted off of the scalp, as its IMU tracks its location continuously. However, it is not advised to move the subject's head because the sensor's 3D position with respect to the head depends upon the head being still.

At the right side, the sensor has detected a pool of conductive fluid within the inner right side of the cranium. This is registered as an anomaly, either by comparison with the other hemisphere, a reading from previous time, or with an average of other readings in the same location from other people. The computer monitor in the figure shows anomaly indication 338 on the right side of the head in head image 330, a little under the sensor graphic 326. Sensor graphic 326 can change color when hovered directly over the anomaly in order to alert the user and indicate that refining measurements are being taken.

The anomaly can be shown as a set of closely overlapping translucent spheres, and they can be in a different color than spheres shown in other detection areas.

In some embodiments, instructions on the computer monitor may direct a user on where and how to place the scanner over the subject. For example, the computer may detect that too few readings were acquired from a front portion of the head and instruct the user to pass over the front of the head again. As another example, comprehensive instructions that take a user from initial calibration all of the way through various scans of the body, rechecking sections, and ending, may also be shown in text or graphical elements. The instructions may direct a user to stop when enough data has been collected.

Once a body portion is scanned, in this case a head, the position of the head and the anomaly may be stored and viewed later for analysis.

A user can rotate the rendering around three orthogonal axes using a mouse, keyboard, touchpad, or other computer input device. Besides rotating the view, the user may also zoom in and out and pan in order to best visualize where the anomaly is in relation to outward physical features of the subject. This is so that the user may better see where the anomaly or anomalies are in order to determine a treatment.

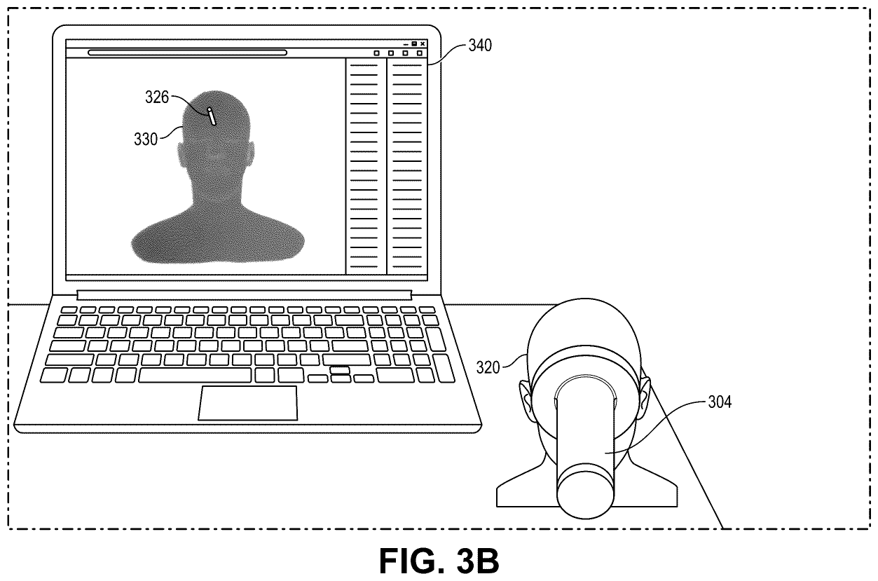

FIG. 4 illustrates monitor 340 displaying an overlayed image from a high-fidelity scan conducted on the patient earlier. MRI image 428 is located and matched with its position on a 3D head model. Two-dimensional MRI image 428 is stretched and turned in order to match the model, and it is overlayed on head image 330. Other MRI or CT scan image slices can be depicted either automatically as the sensor is slewed in azimuth and elevated or by specific commands from the user. Meanwhile, sensor graphic 326 continues to depict the real sensor's position with respect to the head in 3D space.

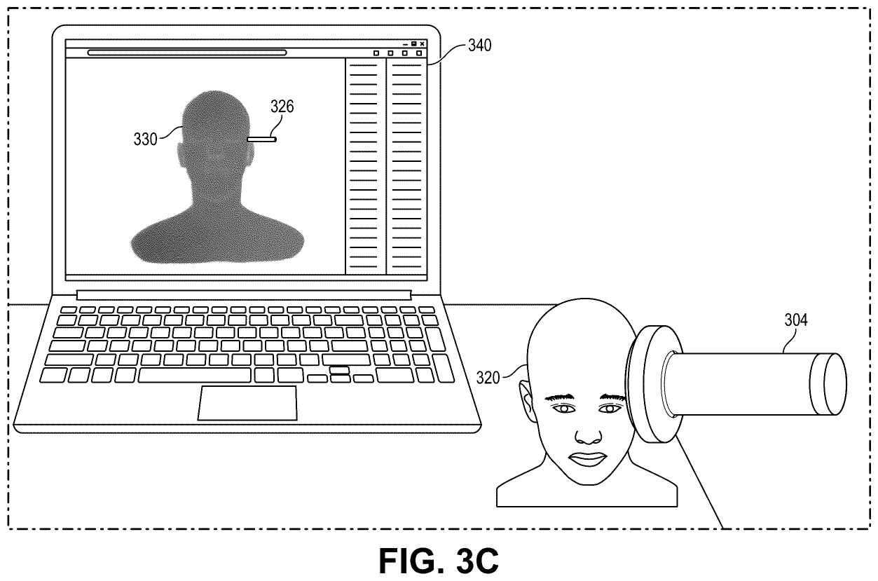

FIG. 5 illustrates a handheld sensor 304 scanning head 320 at a diagonal angle. On display 340, sensor graphic 326 is shown at the same location and diagonal orientation on head image 330 from the head's 3D model. The coil sensor can be moved around a crown of the head in different patterns in order to search for anomalies in real-time.

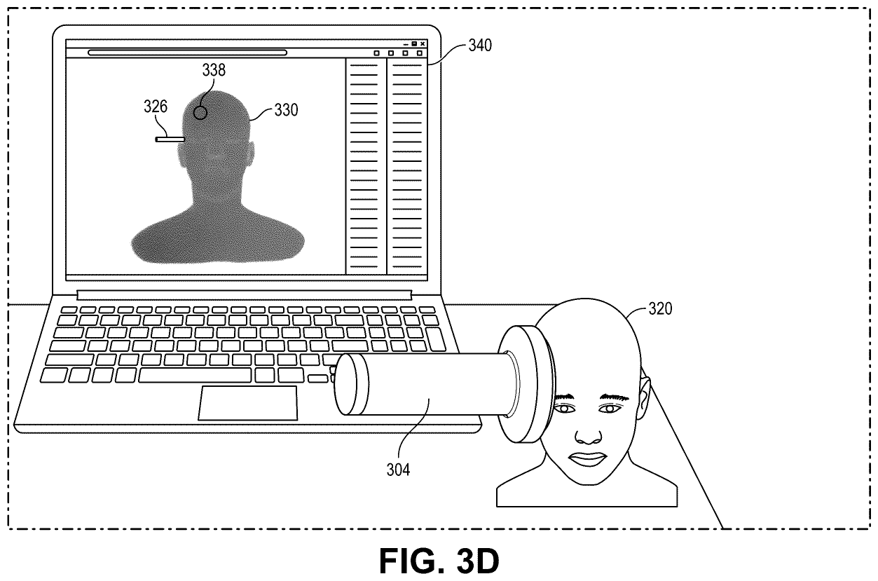

FIG. 6 illustrates MRI images overlayed with a contemporaneous position of the sensor of FIG. 5. Position markers for the sensor, specifically a point on the coil's axis that intersects a front plane of the coil housing, are shown as cross markers. Axial plane MRI image 632 is overlaid with marker 642. Coronal plane MRI image 634 is overlaid with marker 644. Sagittal plane MRI image 636 is overlayed with marker 646. Markers 642, 644, and 646 all indicate a single point in 3D space.

When an anomaly is detected by sensor 304, its location may be indicated on the MRI images. The anomaly can be shown as a translucent sphere or other shape with a size or pattern that indicates the sensor's detection tolerances. The shape may be rendered more opaque or with a brighter hue as further readings confirm a location and definitiveness of the anomaly.

As device 304 (see FIG. 5) is slewed around the head, markers 642, 644, and 646 move on their respective MRI images to show where the target point is. From this, a treating physician may focus on particular areas of interest while scanning.

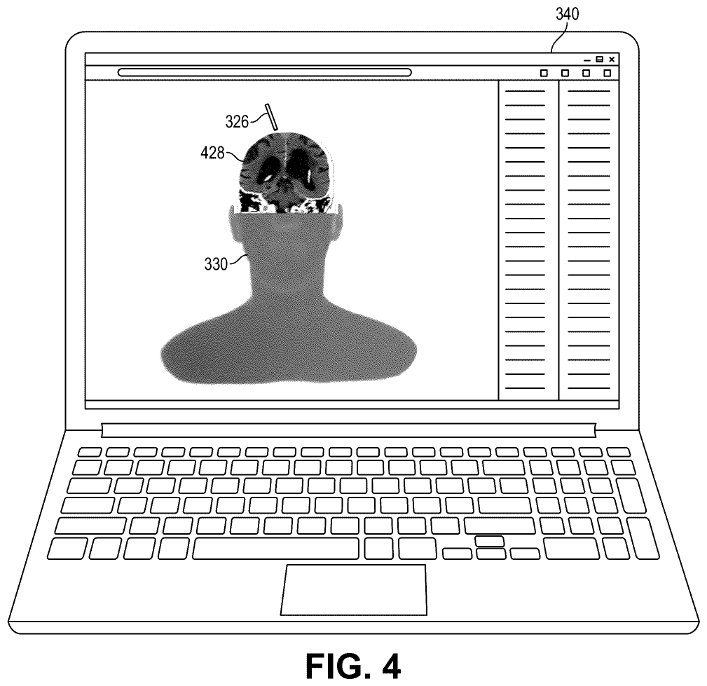

FIG. 7 illustrates a top side perspective view of handheld sensor 704 with aperture 750. Aperture hole 750 runs axially through the handle and coil of the sensor and is wide enough that a surgical tool may be passed through it. That is, both the housings for the handle and coil have a hole passing through them.

In some embodiments, the cylindrical handle of the sensor can be separated from the coil portion. With just the coil portion left on the body, this can provide better visibility and access to the target point on the body.

External ventricular drain (EVD) 752 is shown axially aligned with aperture 750. It is more than narrow enough to be passed through hole 750. When the handle is removed, an incision and borehole into the body may be made more easily. Then, the handle may be reattached.

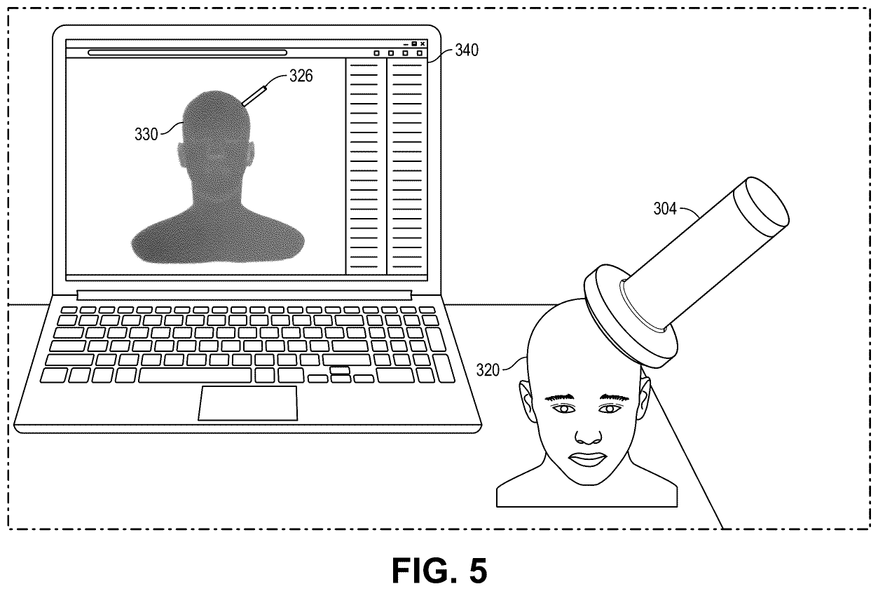

FIG. 8 illustrates sensor device 704 set at a position on cranium 820. What is depicted is a coronal plane of the brain. EVD 752 is being passed through the hole in the device, through the cranium, and into the brain. The distal end of EVD 752 is situated within a ventricle of the brain. Knowing where the distal end of the EVD is in real time can help a ensure that it is in the best possible spot to drain fluid.

Sensor device 704 is commanded into a different mode than one that finds anomalies in nearby tissue. The command may be explicitly sent by the user, automatically switched when the handle is removed from the coil, or otherwise changed in software. This mode counts or measures conductive markers on any compatible surgical tool that passes through the coil. The resonant frequencies of the sensor coil change drastically when conductive materials pass through the coil's center. The purpose is to determine an extent that the tool passes through the coil and therefore estimate the surgical tool's position. For brain surgery, it is effective for internal neuronavigation at a sub-centimeter scale.

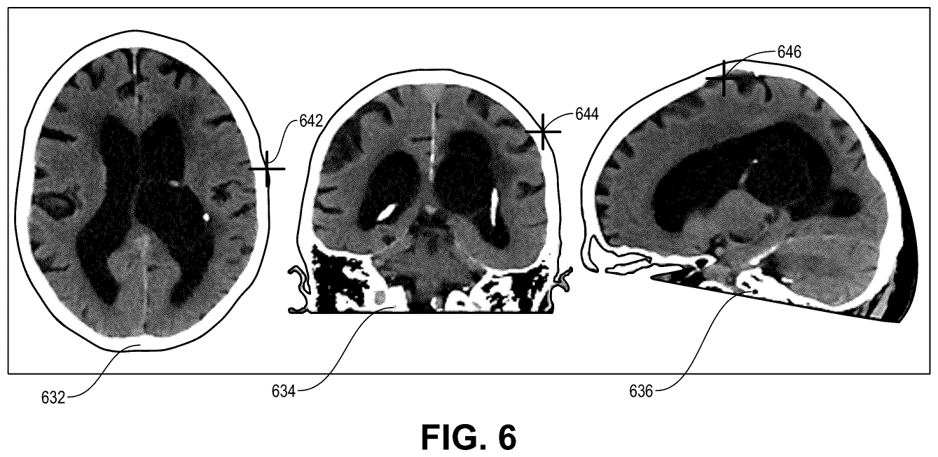

FIG. 9 depicts MRI images 632, 634, and 636 overlayed with a contemporaneous position of EVD 754. Linear graphic 954 depicts EVD 754 in each of the MRI image planes at the appropriate location and orientation. As the EVD is inserted or withdrawn into the brain, line graphic 954 updates to show its extent.

The sensor may output a 3D point where it estimates the distal end of the surgical tool is, or it may output data from which that estimation can be derived. Software can then render the tool as a line on a display, ending at the estimated location. This position may be correlated with position markers on the tool itself or other depth determination techniques.

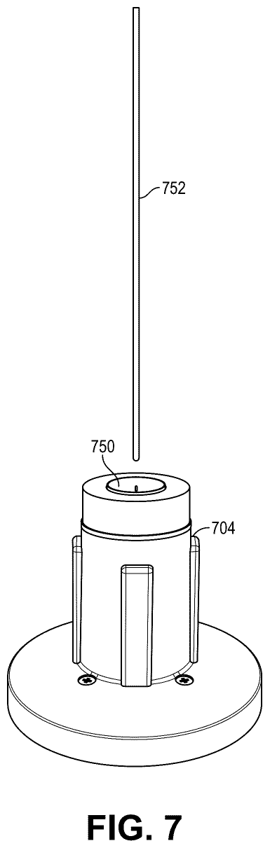

FIGS. 10-12 illustrate EVDs with different markers for determining how far it has passed through a sensor coil. The markers can go on the catheter itself or on the catheter stylet. The same marker concepts can be used on other types of medical devices.

FIG. 10 illustrates EVD 1052 that has metal rings 1054 that are evenly spaced along its longitudinal length. The device can determine how far EVD 1052 has passed through its coil by counting how many rings it has detected passing through it. The rings can extend such that their axes are aligned longitudinally or laterally with the tool.

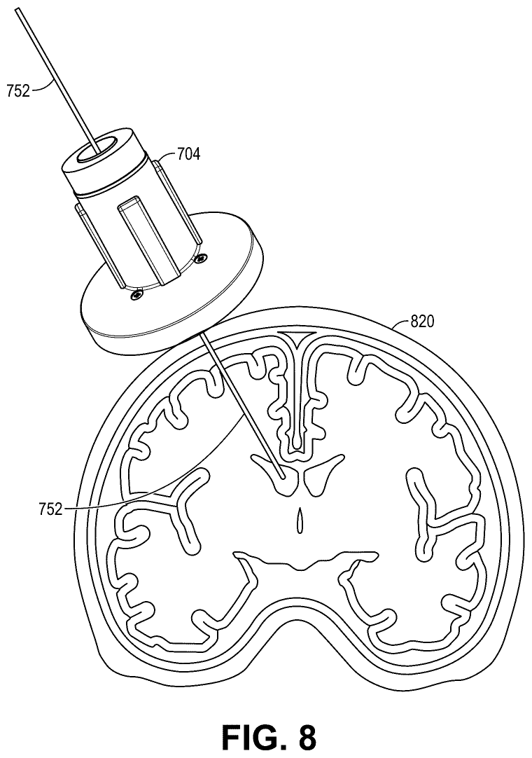

FIG. 11 illustrates EVD 1152 that has rings comprised of different metals 1156, 1158, and 1160 spaced along its longitudinal length. The metals can be aluminum, copper, nickel, platinum, titanium, or other conductive metals. The rings can also be made of nonmetallic conductors, such as graphene. The device can determine how far EVD 1152 has passed through its coil by counting and determining the conductivity of the particular rings that have passed through it. The rings can extend such that their axes are aligned longitudinally or laterally with the tool.

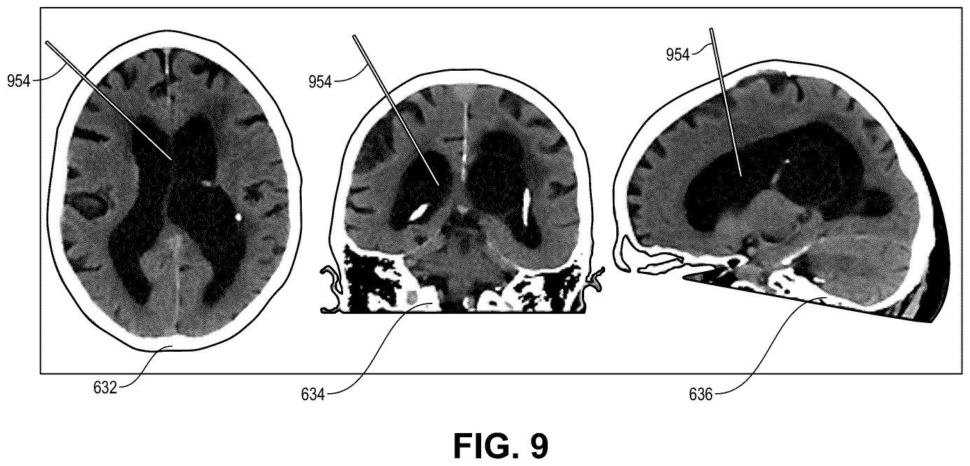

FIG. 12 illustrates EVD 1252 that has catheter with increasing conductor thickness along its length. It is formed as tapered metal piece 1262, thinner distally and getting thicker proximally. The device can determine how far EVD 1252 has passed through its coil by measuring the amount of conductivity of the conductor that has passed through it.

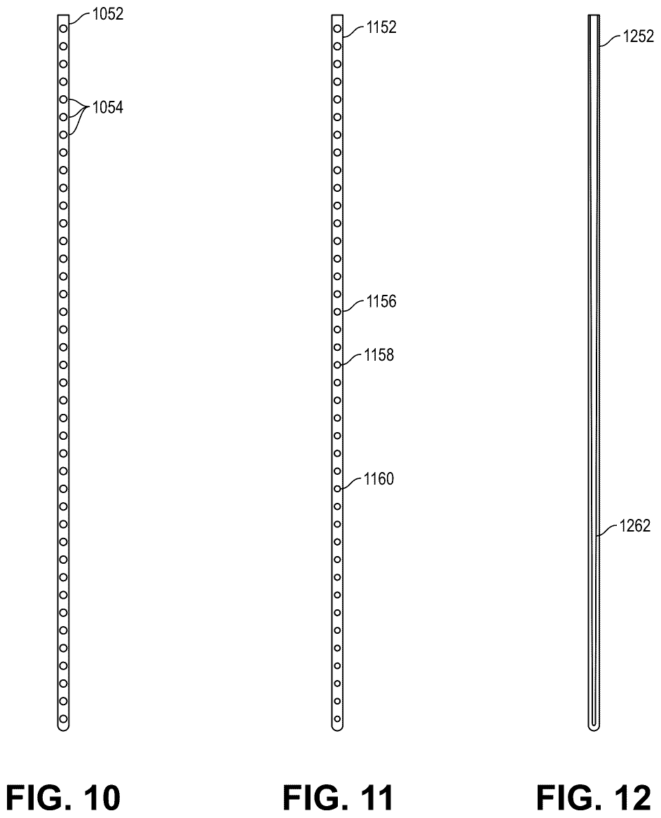

FIG. 13 shows a block diagram of example computer system 1300 for computations. It may utilize any suitable number of subsystems. In some embodiments, a computer system includes a single computer apparatus, where the subsystems can be the components of the computer apparatus. In other embodiments, a computer system can include multiple computer apparatuses, each being a subsystem, with internal components. A computer system can include desktop and laptop computers, tablets, mobile phones and other static and mobile devices.

The subsystems shown in the figure are interconnected via a system bus 1306. Additional subsystems such as a printer 1304, keyboard 1310, storage device(s) 1311, monitor 1308 (e.g., a display screen, such as an LED (light emitting diode) screen), which is coupled to display adapter 1307, and others are shown. Peripherals and input/output (I/O) devices, which couple to I/O controller 1301, can be connected to the computer system by any number of means known in the art such as input/output (I/O) port 1309 (e.g., USB, Fire Wire®). For example, I/O port 1309 or external interface 1312 (e.g., Ethernet, Wi-Fi, etc.) can be used to connect computer system 1300 to a wide area network such as the Internet, a mouse input device, or a scanner. The interconnection via system bus 1306 allows the central processor 1303 to communicate with each subsystem and to control the execution of a plurality of instructions from system memory 1302 or the storage device(s) 1311 (e.g., a fixed disk, such as a hard drive, or optical disk), as well as the exchange of information between subsystems. The system memory 1302 and/or the storage device(s) 1311 may embody a computer readable medium. Another subsystem is a data collection device 1305, such as a camera, microphone, accelerometer, and the like. Any of the data mentioned herein can be output from one component to another component and can be output to the user.

A computer system can include a plurality of the same components or subsystems, e.g., connected together by external interface 1312, by an internal interface, or via removable storage devices that can be connected and removed from one component to another component. In some embodiments, computer systems, subsystem, or apparatuses can communicate over a network. In such instances, one computer can be considered a client and another computer a server, where each can be part of a same computer system. A client and a server can each include multiple systems, subsystems, or components.

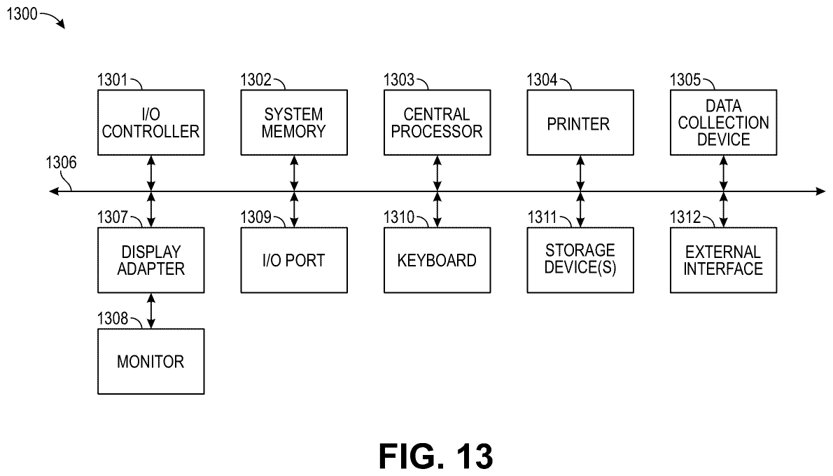

FIG. 14 is a flowchart illustrating process 1400 in accordance with an embodiment. In operation 1401, an electrical coil is provided. In operation 1402, a resistive, inductive, and capacitive (RLC) circuit is electrically connected with the coil. In operation 1403, a frequency counter is electrically connected with the RLC circuit. In operation 1404, an inertial measurement unit (IMU) is rigidly connected with the coil. In operation 1405, a computer processor is connected with a machine-readable non-transitory medium embodying information indicative of instructions for causing a computer processor to perform operations comprising: generating a measured value based on an output from the frequency counter; calculating a position and an orientation of the coil with respect to a body part of a subject based on output from the IMU; and associating the position and orientation with the measured value.

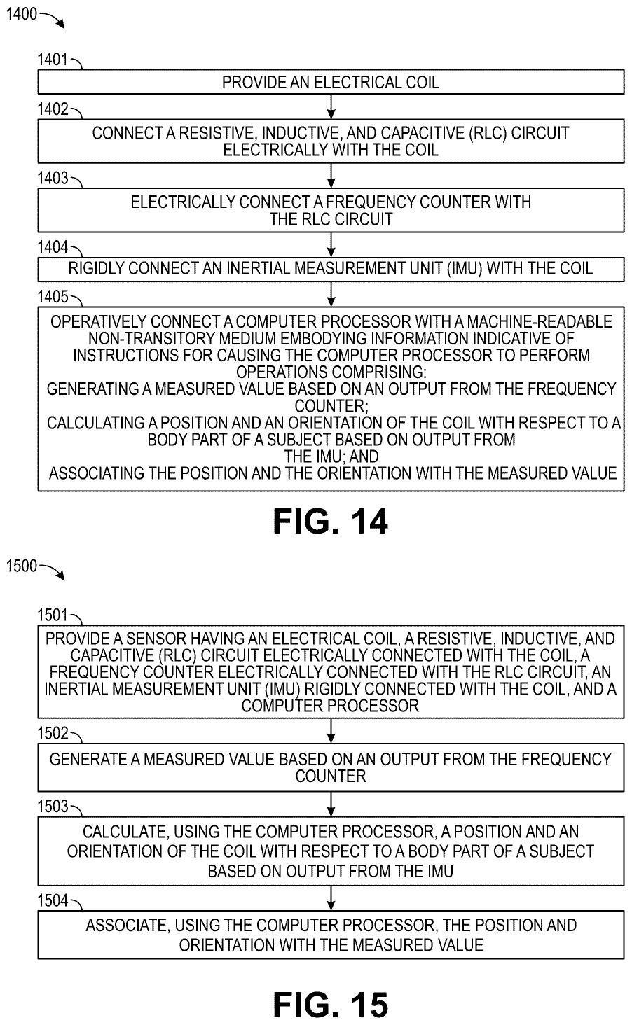

FIG. 15 is a flowchart illustrating a process in accordance with an embodiment. In operation 1501, a sensor having an electrical coil, a resistive, inductive, and capacitive (RLC) circuit electrically connected with the coil, a frequency counter electrically connected with the RLC circuit, an inertial measurement unit (IMU) rigidly connected with the coil, and a computer processor are provided. In operation 1502, a measured value is generated based on an output from the frequency counter. In operation 1503, a position and orientation of the coil with respect to a body part of a subject is calculated based on output from the IMU. In operation 1504, the position and orientation are associated with the measured value using the computer processor.

While the foregoing has described what are considered to be the best mode and/or other examples, it is understood that various modifications may be made therein and that the subject matter disclosed herein may be implemented in various forms and examples, and that the teachings may be applied in numerous applications, only some of which have been described herein. It is intended by the following claims to claim any and all applications, modifications and variations that fall within the true scope of the present teachings.

Unless otherwise stated, all measurements, values, ratings, positions, magnitudes, sizes, and other specifications that are set forth in this specification, including in the claims that follow, are approximate, not exact. They are intended to have a reasonable range that is consistent with the functions to which they relate and with what is customary in the art to which they pertain. “About” in reference to a temperature or other engineering units includes measurements or settings that are within ±1%, ±2%, ±5%, ±10%, or other tolerances of the specified engineering units as known in the art.

The scope of protection is limited solely by the claims that now follow. That scope is intended and should be interpreted to be as broad as is consistent with the ordinary meaning of the language that is used in the claims when interpreted in light of this specification and the prosecution history that follows and to encompass all structural and functional equivalents.

Except as stated immediately above, nothing that has been stated or illustrated is intended or should be interpreted to cause a dedication of any component, step, feature, object, benefit, advantage, or equivalent to the public, regardless of whether it is or is not recited in the claims.

It will be understood that the terms and expressions used herein have the ordinary meaning as is accorded to such terms and expressions with respect to their corresponding respective areas of inquiry and study except where specific meanings have otherwise been set forth herein. Relational terms such as first and second and the like may be used solely to distinguish one entity or action from another without necessarily requiring or implying any actual such relationship or order between such entities or actions. The terms “comprises,” “comprising,” or any other variation thereof, are intended to cover a non-exclusive inclusion, such that a process, method, article, or apparatus that comprises a list of elements does not include only those elements, but may include other elements not expressly listed or inherent to such process, method, article, or apparatus. An element proceeded by “a” or “an” does not, without further constraints, preclude the existence of additional identical elements in the process, method, article, or apparatus that comprises the element.

The Abstract is provided to allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in various embodiments for the purpose of streamlining the disclosure. This method of disclosure is not to be interpreted as reflecting an intention that the claimed embodiments require more features than are expressly recited in each claim. Rather, as the following claims reflect, inventive subject matter lies in less than all features of a single disclosed embodiment. Thus, the following claims are hereby incorporated into the Detailed Description, with each claim standing on its own as a separately claimed subject matter.

What is claimed is:

1. A handheld inductive sensor apparatus for body diagnostics, the apparatus comprising:
an electrical coil;
a resistive, inductive, and capacitive (RLC) circuit electrically connected with the coil;
a frequency counter electrically connected with the RLC circuit;
an inertial measurement unit (IMU) rigidly connected with the coil; and
a computer processor operatively connected with a machine-readable non-transitory medium embodying information indicative of instructions for causing the computer processor to perform operations comprising:
generating a measured value based on an output from the frequency counter;
calculating a position and an orientation of the coil with respect to a body part of a subject based on output from the IMU; and
associating the position and the orientation with the measured value.

2. The apparatus of claim 1 wherein the operations further comprise:
accessing a 3-dimensional (3D) model representing the body part; and
rendering, using the model, an image of the body part along with a graphic representing the position and the orientation of the coil with respect to the body part.

3. The apparatus of claim 2 wherein the model is derived from a computerized tomography (CT) scan or a magnetic resonance imaging (MRI) scan of the body part of the subject.

4. The apparatus of claim 3 wherein the operations further comprise:
depicting, in the image, an internal feature of the body part based on the MRI scan or CT scan.

5. The apparatus of claim 2 wherein the model is of a representatively standard body part of the subject's species.

6. The apparatus of claim 2 wherein the operations further comprise:
determining an anomaly in the body part based on the measured value; and
placing, in the image of the body part, an indicator of the anomaly.

7. The apparatus of claim 2 wherein the operations further comprise:
receiving a calibration command from a user while the coil is placed against the body part at a predetermined location and a predetermined orientation.

8. The apparatus of claim 2 further comprising:
a housing for the coil, the housing including an aperture extending through the coil.

9. The apparatus of claim 8 wherein the operations further comprise:
receiving a command to switch modes;
determining an extent that a surgical tool projects through the aperture based on the measured value; and
depicting, on the image, a depth of the surgical tool within the body part.

10. A surgical kit comprising:
the apparatus of claim 9;
the surgical tool, the surgical tool having conductive rings spaced along a length of a catheter,
wherein the operations further comprise:
counting a number of rings that have passed through the coil; and
calculating the extent based on the counting.

11. A surgical kit comprising:
the apparatus of claim 9;
the surgical tool, the surgical tool having different metals spaced along a length of a catheter,
wherein the operations further comprise:
determining at least one of the metals that has passed through the coil based on a conductivity of the at least one of the metals; and
calculating the extent based on the determining.

12. A surgical kit comprising:
the apparatus of claim 9;
the surgical tool, the surgical tool having an increasing amount of conductive material spaced along a length of a catheter,
wherein the operations further comprise:
determining an amount of conductive material that has passed through the coil; and
calculating the extent based on the determining.

13. The apparatus of claim 1 further comprising:
a proximity sensor connected with the processor,
wherein the operations further comprise:
determining whether the coil abuts the body part based on an output from the proximity sensor; and
locating the body part in 3-dimensional (3D) space based on the determination.

14. The apparatus of claim 13 wherein the operations further comprise:
interpolating a surface in 3D space of the body surface based on the locating of the body part.

15. The apparatus of claim 1 further comprising:
a removable hand grip connected with the coil.

16. The apparatus of claim 1 wherein the operations further comprise:
indicating, based on the measured value, that the coil has moved away from the subject.

17. The apparatus of claim 1 wherein the body part is a head of the subject.

18. The apparatus of claim 17 further comprising:
a housing for the coil, the housing including a recess configured to mate with a nasal bridge of the subject during calibration.

19. A method of manufacturing a handheld inductive sensor for body diagnostics, the method comprising:
providing an electrical coil;
connecting a resistive, inductive, and capacitive (RLC) circuit electrically with the coil;
electrically connecting a frequency counter with the RLC circuit;
rigidly connecting an inertial measurement unit (IMU) with the coil; and
operatively connecting a computer processor with a machine-readable non-transitory medium embodying information indicative of instructions for causing the computer processor to perform operations comprising:
generating a measured value based on an output from the frequency counter;
calculating a position and an orientation of the coil with respect to a body part of a subject based on output from the IMU; and
associating the position and the orientation with the measured value.

20. A method of using a handheld inductive sensor for body diagnostics, the method comprising:
providing a sensor having an electrical coil, a resistive, inductive, and capacitive (RLC) circuit electrically connected with the coil, a frequency counter electrically connected with the RLC circuit, an inertial measurement unit (IMU) rigidly connected with the coil, and a computer processor;
generating a measured value based on an output from the frequency counter;
calculating, using the computer processor, a position and an orientation of the coil with respect to a body part of a subject based on output from the IMU; and
associating, using the computer processor, the position and orientation with the measured value.
